(19) United States
(12) Patent Application Publication
(10) Pub. No.: US 2024/0315720 A1
(43) Pub. Date: Sep. 26, 2024

(54) SLIDER AND SURGICAL INSTRUMENT

(71) Applicant: Riverfield Inc., Tokyo (JP)

(72) Inventor: Koki SHINDO, Tokyo (JP)

(73) Assignee: Riverfield Inc., Tokyo (JP)

(21) Appl. No.: 18/668,504

(22) Filed: May 20, 2024

Related U.S. Application Data
(63) Continuation of application No. PCT/JP2021/043244, filed on Nov. 25, 2021.

(51) Int. Cl. A61B 17/29; A61B 34/00; A61B 34/37

(52) U.S. Cl. CPC A61B 17/2909; A61B 34/37; A61B 34/71; A61B 2017/2924

(57) ABSTRACT

A slider includes a slider body movable in a linear direction with respect to a casing of a surgical instrument, and a roller between the casing and the slider body. The roller is rotatable with respect to the casing and the slider body.

### CROSS-REFERENCE TO RELATED APPLICATIONS

This application n is a continuation of International Application No. PCT/JP2021/043244, filed on Nov. 25, 2021 in the Japan Patent Office, the contents of which being incorporated by reference herein in its entirety.

### BACKGROUND

The present disclosure relates to a slider and a surgical instrument that are used in a master-slave type surgical robot.

In regard to master-slave type surgical robots, there have been demands for a technique to transmit external forces acting on robotic forceps to operators who operate the robots in isolated places in order to improve safety and shorten the time for doctors to learn the operation. The external forces to be transmitted to the operators are estimated based on information such as positions of actuators and driving forces. Hereinafter, robotic forceps that a surgical robot includes may also referred to as a “surgical instrument”. Estimating external forces to be transmitted to an operator may also referred to as a “force sense estimation”.

### SUMMARY

It is an aspect to provide a novel technology for an improved slider and surgical instrument that both improves safety and shortens the time for doctors to learn the operation.

According to an aspect of one or more embodiments, there is provided a slider including: a slider body movable in a linear direction with respect to a casing of a surgical instrument; and a roller between the casing and the slider body being rotatable with respect to the casing and the slider body.

According to another aspect of one or more embodiments, there is provided a surgical instrument including: a casing; a slider body movable in a linear direction with respect to the casing; and a roller between the casing and the slider body, the roller being rotatable with respect to the casing and the slider body.

### BRIEF DESCRIPTION OF THE DRAWINGS

FIG. 1 is a perspective view of a surgical instrument according to one or more embodiments.

FIG. 2 is a perspective view of an internal configuration of the surgical instrument of FIG. 1 according to one or more embodiments.

FIG. 3 is a perspective view of an arrangement of a slider of the surgical instrument of FIG. 1 according to one or more embodiments.

FIG. 4 is an exploded perspective view of the slider of FIG. 3 according to one or more embodiments.

FIG. 5 is a sectional view of a roller, a casing, and a holder according to one or more embodiments.

FIG. 6 is a sectional view of the roller according to one or more embodiments.

FIG. 7 is a sectional view of the roller according to one or more embodiments.

### DETAILED DESCRIPTION

As discussed above, maser-slave type surgical robots have been used. For example, when driving a surgical instrument of a robot, the driving force generated by the driving source, such as an actuator, is transmitted through a slider or a wire. The surgical instrument is then driven from the driving force that is transmitted through the slider or wire.

The slider and the like are subjected to a friction force, such as a static friction force and a kinetic friction force, between the slider and a casing surrounding the slider. In the force sense estimation, information on the position of the actuator or the driving force and the like is used to estimate an external force. Accordingly, the friction force acting on the slider and the like affects the force sense estimation.

Additionally, when the driving force to be transmitted is less than the static friction force, the position of the actuator remains unchanged. This configuration causes a concern because the force sense estimation becomes difficult when the surgical instrument is operated with the driving force being less than the static friction force. In other words, there is a concern that the friction force reduces accuracy of the force sense estimation.

It is an aspect to provide a slider and a surgical instrument that can reduce a loss caused by friction occurring when a driving force is transmitted and inhibit deterioration in accuracy of force sense estimation by having the roller being arranged between the casing and the slider body.

According to some embodiments, a slider may include a slider body movable in a linear direction with respect to a casing of a surgical instrument, and a roller between the casing and the slider body being rotatable with respect to the casing and the slider body.

According to some embodiments, a surgical instrument may include a casing, a slider body movable in a linear direction with respect to the casing, and a roller between the casing and the slider body, the roller being rotatable with respect to the casing and the slider body.

The roller may have a cylindrical or columnar shape and may be supported on the slider body to be rotatable about a rotation axis orthogonal to the linear direction. In a state in which the slider body moves in the linear direction, the roller may be configured to roll while being in contact with the casing.

The roller may have a spherical shape and may be supported on the slider body to be rotatable about a rotation axis orthogonal to the linear direction. In a state in which the slider body moves in the linear direction, the roller may be configured to roll while being in contact with the casing.

Accordingly, a friction force between the casing and the slider body is easily reduced.

In addition, since a static friction force is easily reduced, it is easier to move the slider body even if a driving force applied to the slider body is small. In other words, a position of an actuator that generates the driving force can be easily changed.

Furthermore, reduction in the friction force stabilizes a kinetic friction force at a certain value more easily, thus making it easy to stabilize the driving force during operation of the slider body, and a deterioration in accuracy of force sense estimation can be inhibited.

According to some embodiments, the casing may include a plurality of grooves, a plurality of wires, and a holder corresponding to a portion of the casing.

According to some embodiments, the plurality of grooves may be elongated holes on an end face of the casing. The plurality of grooves may be arranged side by side at equal intervals.

According to some embodiments, the surgical instrument may further include a plurality of sliders, each comprising the slider body and the roller, a multi-degree of freedom manipulator, a joint, and a forceps. The plurality of sliders may be detachably attached to the multi-degree of freedom manipulator. The joint and the forceps may be connected to the casing. The plurality of sliders may be configured to receive a driving force from the multi-degree of freedom manipulator and transmit the driving force to the joint, the forceps, or the joint and the forceps.

According to some embodiments, a number of grooves of the plurality of grooves may correspond to a number of motions of the multi-degree of freedom manipulator. A number sliders of the plurality of sliders may correspond to the number of grooves of the plurality of grooves.

According to some embodiments, the plurality of sliders may be arranged on the plurality of grooves or a portion of the plurality of grooves and moveable in the linear direction with respect to the casing. The plurality of wires may be configured to transmit the driving force received by the plurality of sliders to the joint, the forceps, or the joint and the forceps.

According to some embodiments, the plurality of wires may be made of stainless steel, pure tungsten, an alloy containing tungsten, or a piano wire.

According to some embodiments, the plurality of sliders may be arranged between the holder and the casing. The holder may include a plurality of slits corresponding to the plurality of grooves. The plurality of sliders may be arranged within the plurality of slits and configured to be moveable in the linear direction.

According to some embodiments, the surgical instrument may further include a plurality of first guide pulleys configured to guide the plurality of wires to the joint and the forceps, the plurality of first guide pulleys being arranged closer to the joint and the forceps than the plurality of sliders, a plurality of second guide pulleys arranged closer to the joint and the forceps than the plurality of sliders, and a plurality of third guide pulleys, having a cylindrical shape, arranged more away from the joint and the forceps than the plurality of sliders and comprising two or more grooves on its cylindrical surface configured to guide the plurality of wires. The plurality of first guide pulleys may be further configured to guide the plurality of wires to the plurality of second guide pulleys, the plurality of wires extending from the plurality of sliders. The plurality of second guide pulleys may be configured to guide the plurality of wires to an inside of the joint and the forceps from the plurality of first guide pulleys. The plurality of third guide pulleys may be configured to allow a wire of the plurality of wires to move between the two or more grooves.

According to some embodiments, the slider may further include a first clamp, a second clamp, and a plurality of fixing members having male screws. The slider body may have a square column shape and may include a plurality of female screw holes corresponding to the male screws of the plurality of fixing members and configured to hold the first clamp and the second clamp together, a plurality of positioning protrusions having a cylindrical shape, protruding from the slider body, and configured to determine relative positions between the first clamp and the second clamp with respect to the slider body, and a plurality of rolling shafts on each side face of the slider body.

According to some embodiments, the first clamp and the second clamp are configured to hold a wire of the casing therebetween. The first clamp and the second clamp may overlap each other and are arranged on a top side of the slider. The first clamp may include an insertion hole being a through hole which the wire is inserted, the insertion hole being located in a center of the first clamp, a plurality of positioning holes having a larger diameter than a diameter of the insertion hole, and a plurality of fixing holes having a larger diameter than the diameter of the insertion hole. The plurality of positioning protrusions may be configured to be inserted into the plurality of positioning holes. The plurality of fixing members may be configured to be inserted into the plurality of fixing holes.

According to some embodiments, the second clamp may include a plurality of second positioning holes, a plurality of second fixing holes, and a plurality of guide holes which the wire is inserted therein, the plurality of guide holes being located on each end of second clamp. The plurality of positioning protrusions may be configured to be inserted into the plurality of second positioning holes. The plurality of fixing members may be configured to be inserted into the plurality of second fixing holes.

According to some embodiments, the roller may be provided in a plurality and supported by the plurality of rolling shafts.

According to some embodiments, each of the plurality of rollers may have a cylindrical or columnar shape and mat be supported on the slider body to be rotatable about a rotation axis orthogonal to the linear direction. In a state in which the slider body moves in the linear direction, at least one of the plurality of rollers may be configured to roll while being in contact with the casing and at least one of the plurality of rollers, other than the least one of the plurality of rollers being in contact with the casing, may be configured to roll while being in contact with a holder of the casing.

According to some embodiments, each of the plurality of rollers may have a spherical shape and may be supported on the slider body to be rotatable about a rotation axis orthogonal to the linear direction. In a state in which the slider body moves in the linear direction, at least one of the plurality of rollers may be configured to roll while being in contact with the casing and at least one of the plurality of rollers, other than the least one of the plurality of rollers being in contact with the casing, may be configured to roll while being in contact with the holder.

Accordingly, a friction force between the casing and the slider body is easily reduced.

In addition, since a static friction force is easily reduced, it is easier to move the slider body even if a driving force applied to the slider body is small. In other words, a position of an actuator that generates the driving force can be easily changed.

Furthermore, reduction in the friction force stabilizes a kinetic friction force at a certain value more easily, thus making it easy to stabilize the driving force during operation of the slider body, and a deterioration in accuracy of force sense estimation can be inhibited.

Various embodiments will now be described with reference to the drawings. Components, members, and processes that are the same as or equivalent to each other illustrated in the drawings are represented by the same reference numerals, and redundant explanation will not be repeated where appropriate for conciseness. The present disclosure is not to limit the various embodiments described herein, but rather the various embodiments are provided as examples, and any feature or any combination of features described in the various embodiments is not necessarily essential.

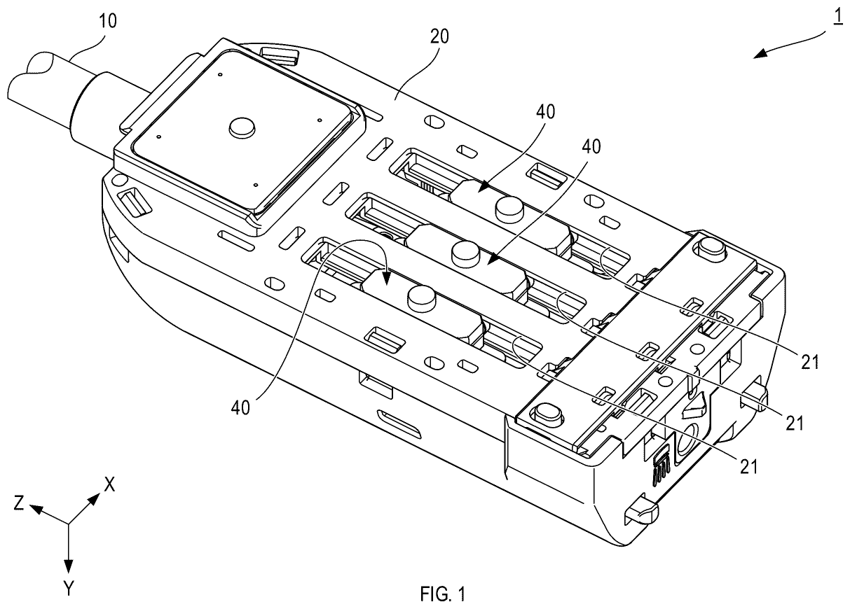

A surgical instrument according to one or more embodiments will be described with reference to FIG. 1 through FIG. 5. A surgical instrument 1 of one or more embodiments is a surgical instrument to be provided on a multi-degree-of-freedom manipulator in a surgical robot that is remotely controllable. The surgical instrument 1 is an instrument used for treatment of a patient. The surgical instrument 1 may include a configuration of forceps or the like used when endoscopic surgery is performed.

As shown in FIG. 1, the surgical instrument 1 may include a shaft 10 and a casing 20.

To simplify the description of the present embodiment, a direction in which the shaft 10 extends is defined as a Z-axis, and a direction toward a leading end from a base of the shaft 10 is defined as a positive direction of the Z-axis. In addition, a direction which is orthogonal to the Z-axis and along which two or more sliders 40 (described below) are aligned is defined as an X-axis, and a left direction when facing in the positive direction of the Z-axis is defined as a positive direction of the X-axis. Further, a direction orthogonal to the Z-axis and the X-axis is defined as a Y-axis, and a direction opposite to the surface where the sliders 40 are aligned in the casing 20 is defined as a positive direction of the Y-axis.

The shaft 10 may be a rod-shaped member to be inserted into a body of a patient. The shaft 10 may be disposed to extend from the casing 20 in the positive direction of the Z-axis. The shaft 10 may have a columnar or a cylindrical shape.

According to one or more embodiments, the shaft 10 may be provided with a joint and forceps at a leading end in the positive direction of the Z-axis.

The joint may be configured to allow changes in orientations of the forceps using the driving force transmitted from the sliders 40 (described below). A specific configuration of the joint may be a general configuration that allows changes in orientations of forceps using a driving force transmitted from the sliders 40, and is not particularly limited.

The forceps may have a configuration similar to general forceps for treatment of a patient. According to one or more embodiments, a case in which the forceps is arranged at the leading end of the shaft 10 is described, but other instruments to be used for treatment of a patient may be arranged at the leading end of the shaft 10.

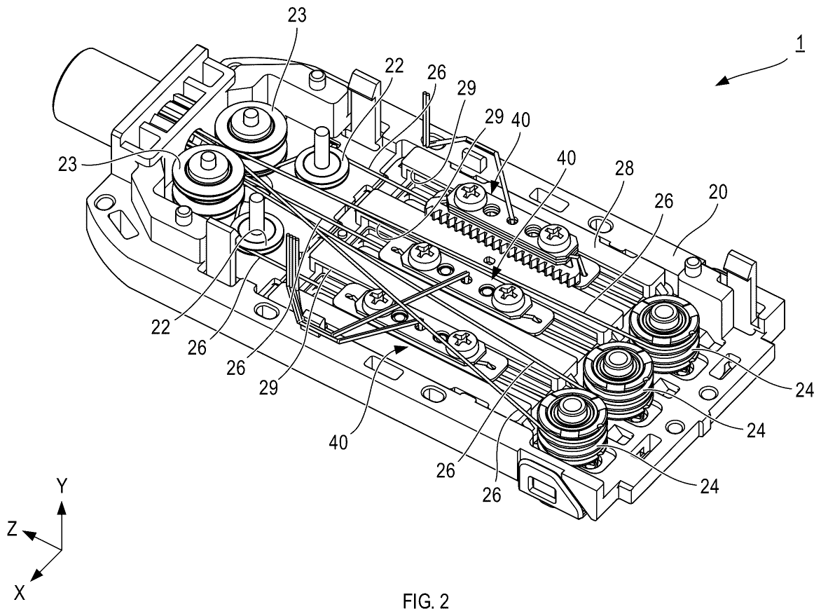

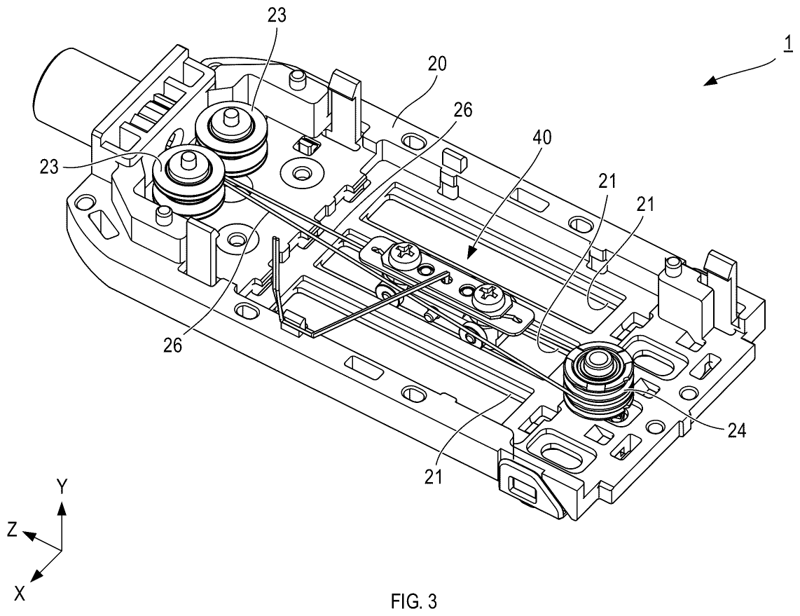

As shown in FIG. 2 and FIG. 3, the casing 20 may be provided with three driven grooves 21, three sliders 40, three wires 26, and one holder 28. The holder 28 corresponds to a portion of the casing.

To simplify the description, FIG. 3 illustrates only the slider 40 and the wire 26 arranged in the central driven groove 21 in the X-axis direction. In FIG. 3, the sliders 40 and wires 26 arranged on the driven grooves 21 located at both ends in the X-axis direction are omitted.

The driven grooves 21 may be elongated holes provided on an end face of the casing 20 located on a negative side in the Y-axis direction. The driven grooves 21 may be elongated holes provided on an attachment/detachment face where a main body 20 and the multi-degree-of-freedom manipulator are attached. Further, the driven grooves 21 may extend along the Z-axis.

The three driven grooves 21 may be arranged side by side at equal intervals in the X-axis direction. The number of the driven grooves 21 may be determined based on motions of the joint, the forceps or the like. In other words, the number of the driven grooves 21 may be determined based on motions in accordance with a specification required for the multi-degree-of-freedom manipulator. The number of the driven grooves 21 may be more than three or less than three, in accordance with the specification required.

The sliders 40 may be configured to receive the driving force from the multi-degree-of-freedom manipulator, and then transmit the driving force to the joint and/or the forceps. The sliders 40 may be detachably attached to the multi-degree-of-freedom manipulator.

One slider 40 may be arranged on each of the three driven grooves 21, one so as to be movable within the driven groove 21 in the Z-axis direction. In other words, the sliders 40 may be arranged so as to be relatively movable in a linear direction with respect to the casing 20. The slider 40 may be arranged on each of all driven grooves 21, or on only a portion of the driven grooves 21.

A portion of the three sliders 40 may be configured to transmit the driving force to the forceps. The rest of the sliders 40 may be configured to transmit the driving force to the joint. For example, two of the three sliders 40 may be configured to transmit the driving force to the forceps. Out of the three sliders 40, one slider 40, other than the sliders 40 transmitting the driving force to the forceps, may be configured to transmit the driving force to the joint.

The wires 26 may be configured to transmit the driving force from the sliders 40 to the forceps or the joint. One or two wires 26 may be arranged on one slider 40.

The wires 26 may each be formed in a long shape. According to one or more embodiments, the wires 26 are made of metal materials such as stainless steel, tungsten, an alloy containing tungsten as an ingredient, and a piano wire, but is not limited thereto.

The holder 28 may be arranged such that the three sliders 40 are arranged between the holder 28 and the casing 20. The holder 28 may be provided with three slits 29 located in positions that correspond to the three driven grooves 21. The slits 29 may be configured to extend along the Z-axis. The sliders 40 may be arranged within the slits 29 so as to be movable in the Z-axis direction within the slits 29.

Further, as shown in FIG. 2, two first guide pulleys 22 that guide the wires 26 to the shaft 10, two second guide pulleys 23, and three third guide pulleys 24 may be provided within the casing 20.

The first guide pulleys 22 may be arranged closer to the shaft 10 than the sliders 40 are. The two first guide pulleys 22 may be arranged side by side, one of which is on a positive side in the X-axis direction in view of the central driven groove 21, and the other may be on a negative side in the X-axis direction in view of the central driven groove 21.

The two first guide pulleys 22 may guide, to the two second guide pulleys 23, the wire 26 extending from the slider 40 located on the positive side in the X-axis direction and the wire 26 extending from the slider 40 located on the negative side in the X-axis direction.

The second guide pulleys 23 may be arranged closer to the shaft 10 than the sliders 40 are, similarly to the first guide pulley 22. The two second guide pulleys 23 may be arranged side by side, one of which is on the positive side in the X-axis direction in view of the central driven groove 21, and the other is on the negative side in the X-axis direction in view of the central driven groove 21.

The two second guide pulleys 23 may guide, to an inside of the shaft 10, the wire 26 extending from the first guide pulley 22 located on the positive side in the X-axis direction and the wire 26 extending from the first guide pulley 22 located on the negative side in the X-axis direction.

The third guide pulleys 24 may be arranged more away from the shaft 10 than the sliders 40 are. The three third guide pulleys 24 may be arranged side by side in the X-axis direction. Each of the third guide pulleys 24 may be formed in a cylindrical shape and includes two or more grooves on its cylindrical surface so as to guide the corresponding wire 26.

According to one or more embodiments, the three grooves may be provided in different positions in the Y-axis direction on the cylindrical surfaces of each third guide pulley 24. The three third guide pulleys 24 may be configured to allow the wire 26 to move from one groove that guides the wire 26 to another groove adjacent thereto.

Out of the three third guide pulleys 24, the third guide pulley 24 on the negative side in the X-axis direction may cause the wire 26 extending from the slider 40 located on the negative side in the X-axis direction to, at first, move from the groove that is guiding the wire 26 to an adjacent groove, so that the wire 26 is differently positioned in the Y-axis direction and then guided to the second guide pulley 23 located on the negative side in the X-axis direction. The second guide pulley 23 on the negative side in the X-axis direction may guide the wire 26 to the inside of the shaft 10.

The guide pulley 24 in the center may cause the wire 26 extending from the slider 40 in the center to, at first, move from the groove that is guiding the wire 26 to an adjacent groove, so that the wire 26 is differently positioned in the Y-axis direction and then guided to the second guide pulley 23 located on the positive side in the X-axis direction. The second guide pulley 23 on the positive side in the X-axis direction may guide the wire 26 to the inside of the shaft 10.

The third guide pulley 24 on the positive side in the X-axis direction may cause the wire 26 extending from the slider 40 located on the positive side in the X-axis direction to, at first, move from the groove that is guiding the wire 26 to an adjacent groove, so that the wire 26 is differently positioned in the Y-axis direction and then guided to the second guide pulley 23 located on the positive side in the X-axis direction. The second guide pulley 23 on the positive side in the X-axis direction may guide the wire 26 to the inside of the shaft 10.

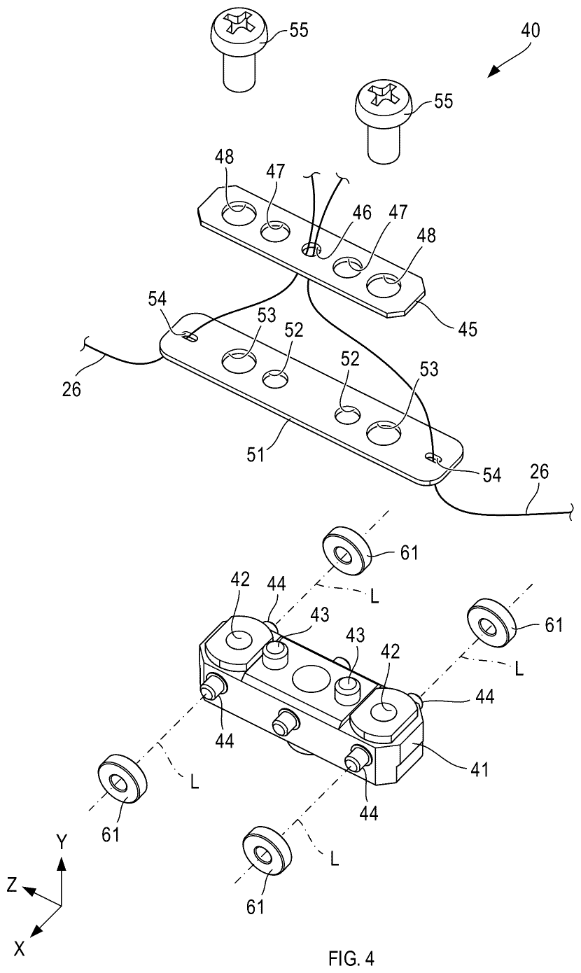

As shown in FIG. 4, the slider 40 may be provided with a slider body 41, one first clamp 45, one second clamp 51, two fixing members 55, and four rollers 61. The slider 40 may comprise an additional member.

The slider body 41 may be a member formed in a pillar shape extending in the Z-axis direction. More specifically, the slider body 41 may be a member formed in a square column shape. The slider body 41 may be provided with two female screw holes 42, two positioning protrusions 43, and four rolling shafts 44.

The female screw holes 42 may match male screws (described below) of the fixing members 55. The female screw holes 42 may be used to hold the first clamp 45 and the second clamp 51 together. The two female screw holes 42 may extend in the Y-axis direction, one of which is at an end part of the slider body 41 in the positive direction of the Z-axis, and the other of which is at an end part of the slider body 41 in the negative direction of the Z-axis.

The positioning protrusions 43 may determine relative positions between the first clamp 45 and the second clamp 51 with respect to the slider body 41. The positioning protrusions 43 may each have a cylindrical shape and protrude from the slider body 41 in the positive direction of the Y-axis. The two positioning protrusions 43 may be provided in respective positions adjacent to the two female screw holes 42 and on a center part of the slider body 41.

The rolling shafts 44 may support the rollers 61 in a rotatable manner about rotation axes L. The rolling shafts 44 may each have a cylindrical shape and protruding from the slider body 41 in the X-axis direction. The center axis of each cylindrical shape is an axis parallel to the X-axis and is a corresponding one of the rotation axes L.

The four rolling shafts 44 may be provided on the slider body 41, two of which are located, in the end part in the positive Z-axis direction, on a side face of the slider body 41 in the positive X-axis direction and a side face thereof in the negative X-axis direction, and the other two of which are located, in the end part in the negative Z-axis direction, on a side face of the slider body 41 in the positive X-axis direction and a side face thereof in the negative X-axis direction.

The first clamp 45 and the second clamp 51 may hold the wire 26 therebetween. The first clamp 45 and the second clamp 51 may be overlapped and arranged on or above a surface of the slider body 41 located on a positive side in the Y-axis direction. The first clamp 45 may be arranged on the positive side in the Y-axis direction, and the second clamp 51 may be arranged on the negative side in the Y-axis direction.

The first clamp 45 may have a rectangular plate-like shape elongated in the Z-axis direction. The first clamp 45 may include one insertion hole 46, two first positioning holes 47, and two first fixing holes 48.

The insertion hole 46 may be a through-hole through which an end of the wire 26 is inserted, and may have a smaller diameter than the first positioning holes 47 and the first fixing holes 48. According to one or more embodiments, the insertion hole 46 may be located in a central area of the first clamp 45 in the Z-axis direction.

The first positioning holes 47 may be through-holes which are each formed to have an inner diameter larger than an outer diameter of each of the positioning protrusions 43 and through which the positioning protrusions 43 are inserted. The two first positioning holes 47 may be formed in respective positions on the first clamp 45 that are opposite to the positioning protrusions 43. According to one or more embodiments, the two first positioning holes 47 may be located in respective positions adjacent to the insertion hole 46, in other words, an adjacent position in the positive Z-axis direction and an adjacent position in the negative Z-axis direction.

The first fixing holes 48 may be through-holes which are each formed to have an inner diameter larger than an outer diameter of each male screw of the fixing members 55 and through which the fixing members 55 are inserted. The first fixing holes 48 may be formed in respective positions opposite to the female screw holes 42.

The two first fixing holes 48 may be formed in respective positions on the first clamp 45 such that the two first positioning holes 47 are arranged between the first fixing holes 48. According to one or more embodiments, the two first fixing holes 48 may be located, one of which is adjacent in the positive Z-axis direction to the first positioning hole 47 located on a positive side in the Z-axis direction, and the other of which is adjacent in the negative Z-axis direction to the first positioning hole 47 located on the negative side in the Z-axis direction.

The second clamp 51 may have a rectangular plate-like shape elongated in the Z-axis direction. The second clamp 51 may have a longer dimension in the Z-axis direction than the first clamp 45 has. The second clamp 51 may include two second positioning holes 52, two second fixing holes 53, and two guide holes 54.

The second positioning holes 52 may be through-holes which are each formed to have an inner diameter larger than the outer diameter of each of the positioning protrusions 43 and through which the positioning protrusions 43 are inserted. The inner diameter of each second positioning hole 52 may be the same as or different from the inner diameter of each first positioning hole 47. The two second positioning holes 52 may be formed in respective positions on the second clamp 51 that are opposite to the positioning protrusions 43.

The second fixing holes 53 may be through-holes which are each formed to have an inner diameter larger than the outer diameter of each male screw of the fixing members 55 and through which the fixing members 55 are inserted. The inner diameter of each second fixing hole 53 may be the same as or different from the inner diameter of each first fixing hole 48.

The two second fixing holes 53 may be formed in respective positions opposite to the female screw holes 42. The two second fixing holes 53 may be formed on the second clamp 51 such that the two second positioning holes 52 are located between the two second fixing holes 53.

According to one or more embodiments, the two second fixing holes 53 may have one located adjacent in the positive Z-axis direction to the second positioning hole 52 located on the positive side in the Z-axis direction, and the other located adjacent in the negative Z-axis direction to the second positioning hole 52 located on the negative side in the Z-axis direction. The guide holes 54 may be through-holes through which the wire 26 is inserted and may be elongated holes extending in the Z-axis direction. The two guide holes 54 may be formed on the second clamp 51 such that the two second fixing holes 53 are located between the two guide holes 54.

According to one or more embodiments, the two guide holes 54 may be located in respective positions, one of which is adjacent in the positive Z-axis direction to the second fixing holes 53 located on the positive side in the Z-axis direction, and the other of which is in an adjacent position in the negative Z-axis direction to the second fixing holes 53 located on the negative side in the Z-axis direction.

The positions where the two guide holes 54 are provided may be contained in an area where the second clamp 51 protrudes more than the first clamp 45 in the positive Z-axis direction and an area where the second clamp 51 protrudes more than the first clamp 45 in the negative Z-axis direction, in a state in which the first clamp 45 and the second clamp 51 are attached to the slider body 41.

The fixing members 55 may be configured to fix the first clamp 45 and the second clamp 51 in such a manner that the first clamp 45 and the second clamp 51 are arranged on or above the slider body 41. In addition, the fixing members 55 may be configured to fix the first clamp 45 and the second clamp 51 with the wire 26 being interposed therebetween. According to one or more embodiments, the fixing members 55 may be male screws.

The rollers 61 may be members that are supported by the rolling shafts 44 in a rotatable manner about the respective rotation axes L. The rollers 61 may each be formed in a cylindrical or columnar shape and configured to have a hole in a center through which the corresponding rolling shaft 44 is inserted.

The four rollers 61 may be arranged on the slider body 41, two of which are located, in the end part in the positive Z-axis direction, on the side face of the slider body 41 in the positive X-axis direction and the side face thereof in the negative X-axis direction, and the other two of which are located, in the end part in the negative Z-axis direction, on the side face of the slider body 41 in the positive X-axis direction and the side face thereof in the negative X-axis direction.

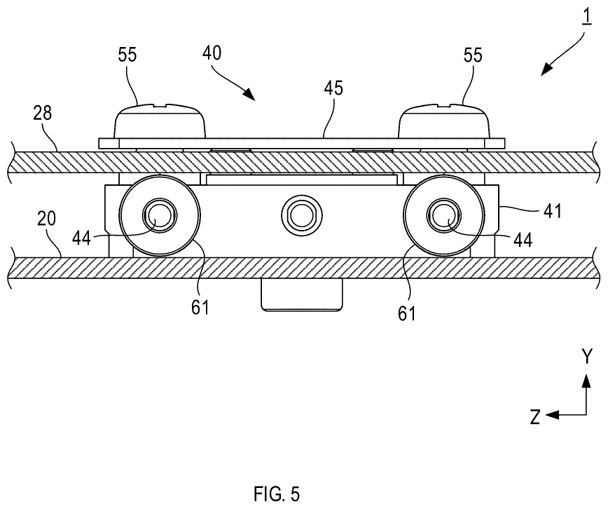

Next, movements of the sliders 40 in the surgical instrument 1 configured as above will be described with reference to FIG. 5.

The driving force to allow motion in the positive direction or negative direction of the Z-axis may be transmitted from the multi-degree-of-freedom manipulator of the surgical robot to the sliders 40. The sliders 40 to which the driving forces have been transmitted linearly may move relative to the casing 20 along the driven groove 21 (see FIG. 3).

The rollers 61 of each slider 40 may have their respective circumferential surfaces in contact with a surface of the casing 20 or a surface the holder 28. When the slider 40 linearly moves relative to the casing, the rollers 61 rotate while in contact with the surface of the casing 20 or the holder 28.

Whether the rollers 61 are in contact with the surface of the casing 20 or the surface of the holder 28 is determined based on an arrangement position of the slider body 41 in the Y-axis direction. The arrangement position of the slider body 41 in the Y-axis direction may be determined based on the arrangement position of the wire 26. The arrangement position of the wire 26 may be determined by which guide pulley, among the first guide pulleys 22, the second guide pulleys 23, and the third guide pulleys 24, guides the wire 26.

All of the four rollers 61 arranged on one slider 40 may be in contact with the surface of the casing 20 or may be in contact with a surface of the holder 28. Alternatively, at least one of the four rollers 61 arranged on one slider 40 may be in contact with the surface of the casing 20, and the rest of the four rollers 61 may be in contact with the surface of the holder 28.

According to the sliders 40 and the surgical instrument 1 as described above, the rollers 61 may be arranged between the slider body 41, and the casing 20 and the holder 28. The rollers 61 may be rotatable relative to the casing 20, the holder 28, and the slider body 41. When the slider body 41 relatively moves in a linear direction with respect to the casing 20 and the holder 28, the rollers 61 rotate while in contact with the casing 20 or the holder 28. Accordingly, a friction force between the casing 20 and the holder 28, and the slider body 41 is easily reduced, and thus a deterioration in accuracy of force sense estimation is easily inhibited.

In addition, since a static friction force is easily reduced, it is easier to move the slider body 41 even if the driving force applied to the slider body 41 is small. In other words, a position of an actuator that generates the driving force can be easily changed, and the surgical instrument 1 can be operated with a smaller driving force. Accordingly, the accuracy of force sense estimation is easily ensured.

The rollers 61 may be supported by the corresponding slider body 41 in a rotatable manner about the respective rotation axes L. Each roller 61 having a cylindrical or columnar shape may rotate while a circumferential surface of the roller 61 is in contact with the surface of the casing 20 or the surface of the holder 28. Accordingly, the friction force between the casing 20 and the holder 28, and the slider body 41 may be easily reduced.

The above-described embodiments are explained in which the rollers 61 each have a cylindrical or columnar shape, but the rollers 61 may have a spherical shape. The slider body 41 is provided with a cylindrical or columnar shape to allow the roller 61 having a spherical shape to rotate.

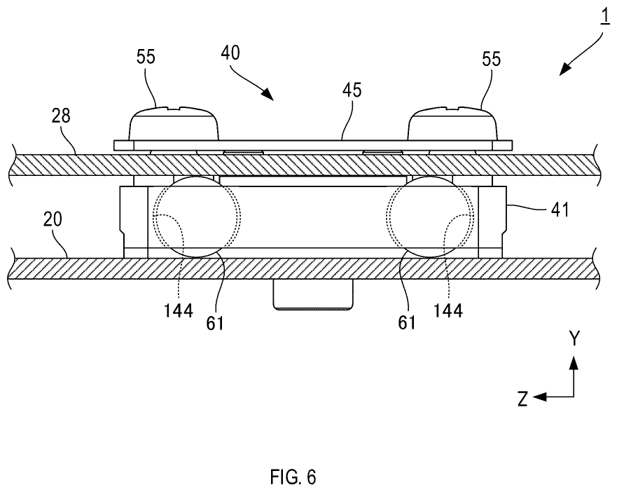

The roller 61 may be arranged recessed portion and a groove. As shown in FIG. 6, a holding portion 144 may hold the sphere-like roller 61 in a rotatable manner while constantly maintaining a relative position with respect to the slider body 41.

Since the roller 61 having a spherical shape is rotatably supported by the slider body 41, the roller 61 may rotate while its spherical surface makes contact with the casing 20. Accordingly, the friction force between the casing 20 and the slider body 41 may be easily reduced.

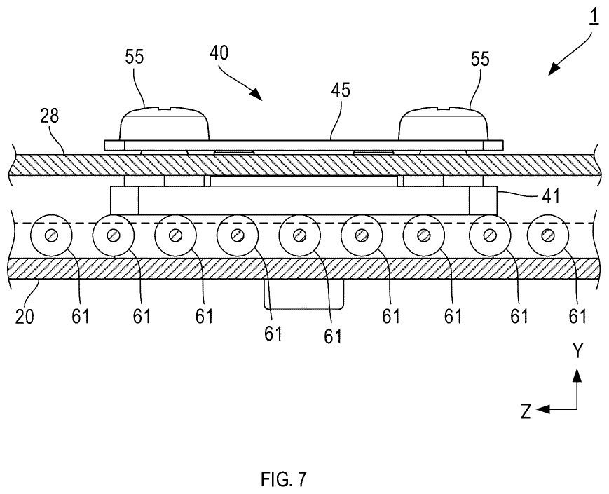

According to one or more embodiments, the roller 61 may be rotatably supported by the casing 20, as shown in FIG. 7.

While various embodiments have been described above with reference to the drawings, the present disclosure is not limited thereto, and any combination or substitution of components as appropriate is included within the scope of the present disclosure. In some embodiments, modifications such as combinations, changes in the order of processes, and various changes in design may be made on the basis of knowledge of a person skilled in the art, and such modified embodiments are within the scope of the present disclosure and the appended claims.

What is claimed is:

1. A slider comprising:
a slider body movable in a linear direction with respect to a casing of a surgical instrument; and
a roller between the casing and the slider body, the roller being rotatable with respect to the casing and the slider body.

2. The slider according to claim 1, wherein the roller has a cylindrical or columnar shape and is supported on the slider body to be rotatable about a rotation axis orthogonal to the linear direction, and
wherein in a state in which the slider body moves in the linear direction, the roller is configured to roll while being in contact with the casing.

3. The slider according to claim 1, wherein the roller has a spherical shape and is supported on the slider body to be rotatable about a rotation axis orthogonal to the linear direction, and
wherein in a state in which the slider body moves in the linear direction, the roller is configured to roll while being in contact with the casing.

4. A surgical instrument comprising:
a casing;
a slider body movable in a linear direction with respect to the casing; and
a roller between the casing and the slider body, the roller being rotatable with respect to the casing and the slider body.

5. The surgical instrument according to claim 4, wherein the roller has a cylindrical or columnar shape and is supported on the slider body to be rotatable about a rotation axis orthogonal to the linear direction, and
wherein in a state in which the slider body moves in the linear direction, the roller is configured to roll while being in contact with the casing.

6. The surgical instrument according to claim 4, wherein the roller has a spherical shape and is supported on the slider body to be rotatable about a rotation axis orthogonal to the linear direction, and
wherein in a state in which the slider body moves in the linear direction, the roller is configured to roll while being in contact with the casing.

7. The surgical instrument according to claim 4, wherein the casing comprises:
a plurality of grooves;
a plurality of wires; and
a holder corresponding to a portion of the casing.

8. The surgical instrument according to claim 7, wherein the plurality of grooves are elongated holes on an end face of the casing, and
wherein the plurality of grooves are arranged side by side at equal intervals.

9. The surgical instrument according to claim 8, further comprising:
a plurality of sliders, each comprising the slider body and the roller;
a multi-degree of freedom manipulator;
a joint; and
a forceps,
wherein the plurality of sliders are detachably attached to the multi-degree of freedom manipulator,
wherein the joint and the forceps are connected to the casing, and
wherein the plurality of sliders are configured to receive a driving force from the multi-degree of freedom manipulator and transmit the driving force to the joint, the forceps, or the joint and the forceps.

10. The surgical instrument according to claim 9, wherein a number of grooves of the plurality of grooves corresponds to a number of motions of the multi-degree of freedom manipulator, and
wherein a number sliders of the plurality of sliders corresponds to the number of grooves of the plurality of grooves.

11. The surgical instrument according to claim 10, wherein the plurality of sliders are arranged on the plurality of grooves or a portion of the plurality of grooves and moveable in the linear direction with respect to the casing, and
wherein the plurality of wires are configured to transmit the driving force received by the plurality of sliders to the joint, the forceps, or the joint and the forceps.

12. The surgical instrument according to claim 7, wherein the plurality of wires comprise stainless steel, pure tungsten, an alloy containing tungsten, or a piano wire.

13. The surgical instrument according to claim 11, wherein the plurality of sliders are arranged between the holder and the casing,
wherein the holder comprises a plurality of slits corresponding to the plurality of grooves, and
wherein the plurality of sliders are arranged within the plurality of slits and configured to be moveable in the linear direction.

14. The surgical instrument according to claim 13, further comprising:
a plurality of first guide pulleys configured to guide the plurality of wires to the joint and the forceps, the plurality of first guide pulleys being arranged closer to the joint and the forceps than the plurality of sliders;
a plurality of second guide pulleys arranged closer to the joint and the forceps than the plurality of sliders; and
a plurality of third guide pulleys, having a cylindrical shape, arranged more away from the joint and the forceps than the plurality of sliders and comprising two or more grooves on its cylindrical surface configured to guide the plurality of wires,
wherein the plurality of first guide pulleys are further configured to guide the plurality of wires to the plurality of second guide pulleys, the plurality of wires extending from the plurality of sliders,
wherein the plurality of second guide pulleys are configured to guide the plurality of wires to an inside of the joint and the forceps from the plurality of first guide pulleys, and
wherein the plurality of third guide pulleys are configured to allow a wire of the plurality of wires to move between the two or more grooves.

15. The slider according to claim 1, further comprising:
a first clamp;
a second clamp; and
a plurality of fixing members having male screws,
wherein the slider body has a square column shape and comprises:
a plurality of female screw holes corresponding to the male screws of the plurality of fixing members and configured to hold the first clamp and the second clamp together;
a plurality of positioning protrusions having a cylindrical shape, protruding from the slider body, and configured to determine relative positions between the first clamp and the second clamp with respect to the slider body; and
a plurality of rolling shafts on each side face of the slider body.

16. The slider according to claim 15, wherein the first clamp and the second clamp are configured to hold a wire of the casing therebetween,
wherein the first clamp and the second clamp overlap each other and are arranged on a top side of the slider,
wherein the first clamp comprises:
an insertion hole being a through hole which the wire is inserted, the insertion hole being located in a center of the first clamp;
a plurality of positioning holes having a larger diameter than a diameter of the insertion hole; and
a plurality of fixing holes having a larger diameter than the diameter of the insertion hole,
wherein the plurality of positioning protrusions are configured to be inserted into the plurality of positioning holes, and
wherein the plurality of fixing members are configured to be inserted into the plurality of fixing holes.

17. The slider according to claim 16, wherein the second clamp comprises:
a plurality of second positioning holes;
a plurality of second fixing holes; and
a plurality of guide holes which the wire is inserted therein, the plurality of guide holes being located on each end of second clamp,
wherein the plurality of positioning protrusions are configured to be inserted into the plurality of second positioning holes, and
wherein the plurality of fixing members are configured to be inserted into the plurality of second fixing holes.

18. The slider according to claim 15, wherein the roller is provided in a plurality, and
wherein the plurality of rollers are supported by the plurality of rolling shafts.

19. The slider according to claim 18, wherein each of the plurality of rollers have a cylindrical or columnar shape and is supported on the slider body to be rotatable about a rotation axis orthogonal to the linear direction, and
wherein in a state in which the slider body moves in the linear direction, at least one of the plurality of rollers is configured to roll while being in contact with the casing and at least one of the plurality of rollers, other than the least one of the plurality of rollers being in contact with the casing, is configured to roll while being in contact with a holder of the casing.

20. The slider according to claim 18, wherein each of the plurality of rollers have a spherical shape and is supported on the slider body to be rotatable about a rotation axis orthogonal to the linear direction, and
wherein in a state in which the slider body moves in the linear direction, at least one of the plurality of rollers is configured to roll while being in contact with the casing and at least one of the plurality of rollers, other than the least one of the plurality of rollers being in contact with the casing, is configured to roll while being in contact with the holder.
